(19) United States
(12) Patent Application Publication
(10) Pub. No.: US 2002/0058824 A1
(43) Pub. Date: May 16, 2002

(54) Solventless synthesis of hydrophilic phenol ester derivatives

(76) Inventors: Victoria Ann Majerczak, Cincinnati, OH (US); Michael Steven Gibson, Cincinnati, OH (US); Robert James Orlando, Cincinnati, OH (US)

Correspondence Address: THE PROCTER & GAMBLE COMPANY, PATENT DIVISION, IVORYDALE TECHNICAL CENTER - BOX 474, 5299 SPRING GROVE AVENUE, CINCINNATI, OH, 45217, US

(21) Appl. No.: 09/998,839

(22) Filed: Nov. 15, 2001

Related U.S. Application Data
(60) Provisional application No. 60/248,924, filed on Nov. 15, 2000.

(51) Int. Cl. C 07C 3 9/12; C07D 317/44

(52) U.S. Cl. 549/438; 554/88; 560/11; 560/41; 560/135

(57) ABSTRACT

Process for preparing benzenesulfonate salts from an appropriately substituted acid chloride and a hydroxybenzenesulfonic acid is conducted in the absence of solvent, in a molar excess of acid chloride and in the presence of a phase transfer catalyst selected from quaternary ammonium and quaternary phosphonium salts and the target reaction product. The molar excess of the acid chloride is used to reduce the occurrence of gelation in the product.

### FIELD OF THE INVENTION

The present invention relates to an improved process for preparing benzenesulfonate salts of the formula (I)

RCO₂ PhSO₃M (I)

from acid chlorides and salts of phenol sulfonic acids in which the reaction is conducted in the presence of a phase transfer catalyst, but substantially free of organic or aqueous solvents.

### DESCRIPTION OF PRIOR ART

U.S. Pat. No. 4,704,236 describes a process for preparing acyloxybenzene sulfonate salts in which an alkali metal phenol sulfonate is reacted with an aliphatic acyl halide at a temperature of from 135° C. to 180° C. in the presence of an organic solvent. Alkali metal acyloxybenzene sulfonate salts precipitate from the reaction mixture as separable solids. It is stated that the aliphatic acyl halide is preferably a linear aliphatic acyl chloride which contains from 6 to 15 carbon atoms, including specifically the acid chlorides derived from heptanoic acid, octanoic acid, nonanoic acid, and decanoic acid. Where branched chain acyl chlorides are used, no difference in yield is noted whether the solvent is aromatic or aliphatic. However, when linear acyl chlorides are used, it is stated in col. 3., lines 7-13, that a very distinct benefit in yield can be achieved when the reaction is carried out in the presence of an aliphatic hydrocarbon solvent. The mole ratio of acyl chloride to alkali metal phenol sulfonate in the examples varies from about 1.5:1 to 2:1.

European Patent Application 0 148 148 describes a process for preparing sodium alkanoyloxyhalidebenzene sulfonates by reacting substantially solid anhydrous sodium phenol sulfonate with alkanoylhalide at a temperature in the range of 90° C. to 200° C. in the substantial absence of a solvent or an inert reaction medium.

European Patent Application 0 164 786 describes a process for preparing p-isononanoyloxybenzenesulfonate by reacting isononanoic acid chloride with potassium p-phenolsulfonate in the presence of a solvent, preferably an aromatic hydrocarbon, at a temperature in the range of 80° C. to 200° C.

U.S. Pat. No. 4,536,314 describes the preparation of branched chain aliphatic peroxyacid bleach precursors, such as, for example, sodium 3,5,5-trimethyl hexanoyloxybenzene sulfonate, which is obtained from the reaction of isononanoyl chloride and anhydrous sodium phenol sulfonate. Example 1 describes the reaction in greater detail. Tetrabutylammonium bromide is added to the reaction mixture as a catalyst, but there is no teaching or explanation as to the need or the desirability for employing a catalyst for this type of reaction. Moreover, the applicability of a catalyst in preparing other than branched chain, i.e., linear, precursors as well as its chemistry are left open to speculation.

U.S. Pat. No. 5,069,828, describes the preparation of benzenesulfonate salts from the reaction of an acid chloride and a hydroxybenzenesulfonic acid in the presence of a phase transfer catalyst selected from quaternary ammonium and quaternary phosphonium salts. The described reaction requires the presence of a solvent, preferably an aprotic solvent such as an aliphatic or an aromatic hydrocarbon or a halogenated aliphatic or aromatic hydrocarbon or mixtures thereof. It is asserted that the combination of the catalyst and the low boiling point solvent accelerates the reaction rate and provides yields with relatively high purity.

Many of the compounds which can be prepared by the process of the present invention are known in the art, especially for their utility as bleach activators. The term “bleach activator” is understood in the art to describe a relatively stable compound which will decompose in water in the presence of a peroxygen to give the corresponding peracid bleaching agent.

The bleach activators which can be prepared by the process of the present invention are described in the references cited above as well as in U.K. Patent Specification No. 864,798, European Patent Application 267,048, European Patent Application 284,292, U.S. Pat. Nos. 4,483,778, 4,536,314, 4,634,551, 4,681,592, 4,778,618 and 4,735,740.

### SUMMARY OF THE INVENTION

The present invention provides an improved process for preparing alkanoyloxybenzenesulfonate salts of the formula (I)

RCO₂PhSO₃M (I)

where:

R is C₁-C₂₀ linear alkyl; C₁-C₁₅ alkyl substituted by N(R₁)COR₂, CONR₁R₂, CO₂ R₃, OR₃ or SO₂ R₃ ; OR₃; CH═CHCO₂R₃; phenyl substituted by CO₂R₃; CH(OR₃)₂; CH(SO₂R₃)₂; C(R₄)(R₅)Cl; C(R₇)₂OC(O)R₆; or CH₂OR₈;

R₁ is H or C₁-C₁₀ alkyl, aryl or alkaryl;

R₂ is C₁-C₁₄ alkyl, aryl or alkaryl;

R₃ is C₁ -C₂₀ alkyl, alkenyl, alkynyl or alkaryl, optionally alkoxylated with one or more ethyleneoxy or propyleneoxy groups or mixtures thereof;

R₄ is C₄-C₁₄ alkyl or alkenyl;

R₅ is H, methyl or ethyl;

R₆ is C₁-C₂₀ linear or branched alkyl, alkylethoxyalkylated, cycloalkyl, aryl or substituted aryl;

R₇ are independently H, C₁-C₂₀ alkyl, aryl, C₁-C₂₀ alkylaryl and substituted aryl;

R₈ is aryl optionally substituted by C₁-C₅ alkyl; and

M is selected from an alkali metal or an alkaline earth metal.

Compounds within this group which are of particular utility as bleach activators, and those for which the process of the invention is particularly applicable, include the compounds where R is C₅-C₉ linear alkyl; C₁-C₄ alkyl substituted by (R₁)COR₂, CONR₁R₂, CO₂R₃, OR₃ or SO₂R₃; OR₃; C(R₇)₂OC(O)R₆; or CH₂OR₈.

The alkanoyloxybenzenesulfonate salts of formula (I) are prepared by reacting an acid chloride (II) with the appropriate salt of phenol sulfonic acid (III) in the presence of a phase transfer catalyst (PTC) as shown in Equation 1, the improvement comprising conducting the reaction in the absence of an aqueous or organic solvent.

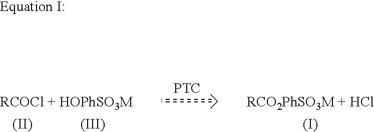

According to the invention, it has unexpectedly been found that the reaction according to Equation 1 can be easily carried out in the absence of a solvent with a molar excess of the acid chloride and the addition of a phase transfer catalyst selected from quaternary ammonium and quaternary phosphonium salts, or the target phenol ester product, in an amount ranging from 0.1 up to 10 weight percent relative to the initial charged weight of the reactants, namely, the acid chloride and the salt of the phenolsulfonic acid. Similar reactions that have been run in the absence of solvents or which have been carried out in a solvent having a low boiling point, generally produce poor yields and frequently give a gel by-product that makes isolation of the product impractical if not impossible.

The process of this invention allows the preparation of alkanoyloxybenzene sulfonate salts of high purity in the absence of solvent with a significantly reduced occurrence of gelation in the product.

### DETAILED DESCRIPTION OF THE INVENTION

The benzenesulfonate salts of formula (I) can be prepared by reacting an acid chloride (II) with a salt of phenol sulfonic acid (III). Acid halides, phenol sulfonic acid salts and phase transfer catalysts suitable for use in the process of this invention are known or they may be prepared by methods known in the art. The terminology “alkali metals” as used herein refers to the Group 1a metals lithium, sodium, potassium, rubidium, and cesium. The terminology “alkaline earth metals” refers to the Group 2a metals beryllium, magnesium, calcium, strontium, and barium.

The reaction according to Equation 1 is to be carried out in the absence of a solvent. Solventless reactions have been avoided in the production of phenol ester derivatives because of poor yields, and in particular because of the gelation that tends to occur and which makes the isolation of the product impractical if not impossible. The process of the present invention overcomes these problems by reacting a sodium phenol sulfonate (“SPS”) in a molar excess of an appropriate acid chloride in the presence of a phase transfer catalyst. Surprisingly, gelation in the final product can be significantly reduced or avoided where the acid chloride is used in molar excess relative to the SPS, preferably in a ratio between about 4.5:1 and about 8:1, preferably at least 5.5:1, and even more preferably at least 6.7:1. Further, the reaction is carried out at moderate temperatures in the range of 90° to 120° C. using a blanket of inert gas and/or sparging with an inert gas to remove the hydrogen chloride (“HCl”) by-product. Preferably, when sparging is used, the flow of gas should be only high enough to continue driving off HCl but low enough to avoid foaming of the reactants. Further, although thorough mixing is required initially, it is preferable to avoid excessive agitation and mixing during the course of the reaction so as to prevent foaming and gelation during the reaction.

According to the present invention, the reaction between an acid chloride and an phenol sulfonic acid salt can be carried out in a solventless reaction without gelation by adding a phase transfer catalyst and using a molar excess of the appropriate acid chloride. The presence of a phase transfer catalyst promotes contact between the reactants and enables the reaction product to puff up into a porous mass. The formation of this porous product enables the hydrogen chloride by-product to evolve off or to be drawn off under vacuum or sparging.

Suitable phase transfer catalysts can be selected from among those described by C. M. Starks and C. Liotta in “Phase Transfer Catalysis, Principles and Techniques” (Academic Press, Inc., N.Y., N.Y., 1978) and among those described by E. V. Dehmlow and S. S. Dehmlow in “Phase Transfer Catalysis, 2nd Ed.” (Verlag Chemie GmbH, D-6940 Weinheim, 1983), the teachings of which are incorporated herein by reference. Quaternary ammonium and quaternary phosphonium salts are particularly useful phase transfer catalysts for practicing the process of this invention. Useful quaternary ammonium and phosphonium salts include, but are not limited to, chlorides, bromides, iodides, fluorides, hydrogen sulfates, sulfates and dihyrogen phosphates. In addition, it has been unexpectedly found that the reaction product itself may be used as the phase transfer catalyst.

Specific quaternary ammonium and phosphonium phase transfer catalysts which can be used according to the improved process of this invention include, but are not limited to, tetramethylammonium bromide, tetramethylammonium chloride, tetramethylammonium hydrogen sulfate, tetramethylammonium sulfate, tetramethylammonium idodide, tetraethylammonium bromide, tetraethylammonium chloride, tetraethylammonium hydrogen sulfate, tetraethylammonium iodide, tetrapropylammonium bromide, tetrapropylammonium chloride, tetrapropylammonium hydrogen sulfate, tetrapropylammonium iodide; methyltriethylammonium bromide, methyltriethylammonium chloride, methyltriethylammonium hydrogen sulfate, methyltriethylammonium iodide, methyltripropylammonium bromide, methyltripropylammonium chloride, methyltripropylammonium hydrogen sulfate, methyltripropylammonium iodide, methyltributylammonium bromide, methyltributylammonium chloride, methyltributylammonium hydrogen sulfate, methyltributylammonium iodide, tetrabutylammonium fluoride, tetrabutylammonium dihydrogenphosphate, tetrabutylammonium bromide, tetrabutylammonium chloride, tetrabutylammonium hydrogen sulfate, methyltrioctylammonium bromide, methyltrioctylammonium chloride, methyltrioctylammonium iodide, octadecyltrimethylammonium bromide, Aliquat.RTM. 336, hexadecyltrimethylammonium bromide, hexadecyltrimethylammonium chloride, benzyltrimethylammonium bromide, benzyltrimethylammonium chloride, benzyltriethylammonium bromide, benzyltriethylammonium chloride, benzyltributylammonium bromide, benzyltributylammonium chloride, tetrabutylphosphonium bromide, tetrabutylphosphonium chloride, hexadecyltributylphosphonium bromide, tetraphenylphosphonium bromide, tetraphenylphosphonium chloride, methyltriphenylphosphonium bromide, and methyltriphenylphosphonium iodide. Particularly preferred catalysts include tetradecyltrimethyl ammonium bromide and tetrabutylammonium bromide.

Chloride salts may be preferred for their high catalytic activity and because of a reduced likelihood of producing undesirable colored by-products. Likewise, because residual amounts of the halide may persist in the final product, chlorides may be preferred over bromides and iodides, because chloride is not as readily oxidized in wash water to a corresponding hypohalite, which, in turn, has been found to cause fabric dye damage under certain conditions.

Any practical amount of catalyst may be employed, but preferably the amount should be between about 0.1 and about 10 weight percent relative to the amount of SPS charged. In a preferred embodiment, the amount of catalyst employed should be from about 1 to 5 mole percent relative to the amount of SPS and acid chloride present in the reaction.

The optimum catalyst and temperature for carrying out the process of this invention will depend on the nature of the SPS (III) and the acid chloride (II) which comprise the starting materials. The order of addition of the starting materials is not critical; however, it is preferable to add the SPS(III) to a stirred mixture of the acid chloride (II) and the catalyst. Although it is not essential, it is advantageous to carry out the reaction under an atmosphere of an inert gas, such as argon or nitrogen.

In cases where the SPS (III) is obtained as a hydrated material, it is beneficial to remove as much water as possible prior to its addition. This may be conveniently accomplished by drying the phenol derivative in a vacuum oven or by azeotropic removal of the water in the presence of an appropriate solvent. Examples of such solvents include aliphatic and aromatic hydrocarbons and halogenated aliphatic and aromatic hydrocarbons, the specific selection of which is within the knowledge of those skilled in the art. One method for preparing the anhydrous SPS is provided in U.S. Pat. No. 4,666,636, issued to Monsanto Company, the description of which is incorporated herein by reference. Regardless of the method used to remove the moisture from the SPS, the SPS used in the reaction with the acid chloride should be anhydrous, having a moisture content less than or equal to about 0.5% to prevent hydrolysis of the acid chloride.

In order to reduce and avoid the potential of gelation in the final product, the reaction according to Equation 1 should be carried out using a molar excess of acid chloride. The ratio of acid chloride to SPS should be between about 4.5:1 and about 8:1. Best results, however, are achieved using at least a 6.7:1 molar ratio of the reactants, contrary to the teachings of the prior art.

The target weight ratio of acid chloride to final product should be about 65:35, with about 70:30 being preferred and about 75:25 being even more preferred. These results may be achieved using a starting mole ratio of acid chloride to sodium phenol sulfonate of about 4.5-5.0:1, of about 5.0-6.0:1 and about 6.0-7.0:1, respectively. The use of excess acid chloride provides a relatively complete conversion of the SPS (III) in a commercially feasible period of time. Unreacted acid chloride (II) may be recovered and reused.

In most cases the product of formula (I) will be a solid at reaction temperatures and at ambient temperature. Preferably, the solid product will be porous to provide fluid communication to facilitate the passage of gas such as the evolution of HCl during the reaction. The product is allowed to cool and optionally washed with an organic solvent and filtered several times to isolate the product. Where desired, the compounds of formula (I) may also be further purified by recrystallization or tritration with water or organic solvents or mixtures thereof. Mixtures of water and alcohols, such as methanol, ethanol and isopropanol, are well suited to this purpose.

The compounds within the scope of formula (I) are useful as bleach activators and as additives for laundry detergents. The process of this invention is further illustrated in the following examples.

### EXAMPLE 1

### Preparation of Branched Nonanoyl Benzene Sulfonate Derivatives

106 g of anhydrous sodium phenol sulfonate was stirred into 100 g of 3,5,5-trimethylhexanoyl chloride under a current of nitrogen. 5 g C₁₄H₂₉N(CH₃)₃⁺BR⁻ was added and the mixture was heated and stirred at 95-100° C. for 4 hours under a current of nitrogen. After 2 hours, the paste puffed up into an off-white porous solid and stirring was discontinued. The mass was allowed to cool and was broken up under ethyl acetate, stirred, filtered, rewashed, refiltered and dried. The yield was 120 g and the purity using NMR was 85%.

### EXAMPLE 2

### Preparation of Nonanoyl Oxybenzene Sulfonate

26.36 g of sodium phenol sulfonate were mixed with 175.99 g of nonanoyl chloride in a 300 L reactor. These amounts reflect a molar ratio of acid chloride to sodium phenol sulfonate in excess of 7:1. 9.12 g of nonanoyl oxybenzene sulfonic acid was added as a catalyst. An Arrow 350 Agitator (Arrow Engineering Co, Inc.-Pennsylvania) with the transformer set at 40 rpm was used to mix the reactants. A small volume of nitrogen sparging was provided. A cold water condenser was provided attached to a scrubber charged with sufficient sodium hydroxide to neutralize the condensed hydrogen chloride. The reactor was heated to 85° C. Gelation began to occur at 0.5 hrs into the reaction but disappeared about 1.0 hrs into the reaction. During the course of the reaction the flow of nitrogen was reduced to a minimum and the stirring was reduced to 30 rpm. The reaction was complete after about 2 hrs. Following isolation, analysis using a Cat SO₃ test revealed a 94.89% purity with no gelling in the final product. Yield was approximately 85%.

### EXAMPLE 3

### Preparation of Nonanoyl Oxybenzene Sulfonate

89.68 g of nonanoyl chloride was charged to a reactor vessel and heated to 95° C. 4.7 g of tetradecyltrimethylammonium bromide was added to the heated acid chloride and allowed to stir for approximately 20 minutes. 69.7 g of sodium phenol sulfonate (anhydrous—0.42% water) was added to the mixture, which gave a mole ratio of 1.44:1 acid chloride to SPS. A nitrogen blanket was used to prevent external moisture from interfering in the reaction. As the reaction proceeded, the product began to puff up as the hydrogen chloride evolved. The reaction was complete 30 minutes after the addition of the sodium phenol sulfonate. An odor of nonanoyl chloride was observed in the product prior to isolation of the nonanoyl oxybenzene sulfonate. Subsequent analysis confirmed a 73.5% conversion to nonanoyl oxybenzene sulfonate.

### EXAMPLE 4

### Preparation of Nonanoyl Oxybenzene Sulfonate

Using the same procedure as in Example 3, 88.69 g of sodium phenol sulfonate was combined with 237.86 g of nonanoyl chloride representing nearly a 3:1 ratio of acid chloride to sodium phenol sulfonate. 2.05 g of nonanoyl oxybenzene sulfonic acid was added to catalyze the reaction. Nitrogen sparging was provided and the reaction vessel was heated to about 80° C. By about 0.5 hrs into the reaction hydrogen chloride was evolving off rapidly so sparging was discontinued. Foaming and gelation were also observed less than 0.5 hrs into the reaction. As reaction began to slow, sparging was resumed and stirring increased to drive the reaction to completion between 1 and 1.5 hrs. Following isolation and analysis, a purity of 99.7% was determined. Yield was approximately 80%.

Contemplated equivalents for the process of this invention are those cases in which the acid chloride (II) is derived from any carboxylic acid so long as the acid is free of functional groups which would interfere with the process. Also included in this group are diacid chlorides such as those derived from C₆-C₂₀ linear dicarboxylic acids. In the same way, the phenyl group of the starting hydroxybenzenesulfonic acid salt may be partially or fully substituted so long as the substituted groups does not interfere with the process.

What is claimed is:

1. A process for preparing a phenol ester derivative of the formula (I)
RCO₂PhSO₃M (I)
where:
R is C₁-C₂₀ linear alkyl; C₁-C₁₅ alkyl substituted by N(R₁)COR₂, CONR₁R₂, CO₂R₃, OR₃ or SO₂R₃; OR₃; CH═CHCO₂R₃; phenyl substituted by CO₂ R₃; CH(OR₃)₂; CH(SO₂R₃)₂; C(R₄)(R₅)Cl; C(R₇)₂OC(O)R₆; or CH₂OR₈;
R₁ is H or C₁-C₁₀ alkyl, aryl or alkaryl;
R₂ is C₁-C₁₄ alkyl, aryl or alkaryl;
R₃ is C₁-C₂₀ alkyl, alkenyl, alkynyl or alkaryl, optionally alkoxylated with one or more ethyleneoxy or propyleneoxy groups or mixtures thereof;
R₄ is C₄-C₁₄ alkyl or alkenyl;
R₅ is H, methyl or ethyl;
R₆ is C₁-C₂₀ linear or branched alkyl, alkylethoxyalkylated, cycloalkyl, aryl or substituted aryl;
R₇ are independently H, C₁-C₂₀ alkyl, aryl, C₁-C₂₀ alkylaryl and substituted aryl;
R₈ is aryl optionally substituted by C₁ -C₅ alkyl; and
M is selected from an alkali metal or an alkaline earth metal,
the process comprising the steps of reacting an acid chloride of the formula (II)
RCOCl (II)
with a phenol sulfonic acid salt of the formula (III)
HOPhSO₃M (III)
in the absence of solvent, and in the presence of from 0.1 to 10 weight percent of a phase transfer catalyst selected from quaternary ammonium compounds, quaternary phosphonium salts and phenol ester derivatives of formula (I), based on the amount of phenol sulfonic acid salt and acid chloride present in the reaction.

2. The process of claim 1, wherein the phase transfer catalyst is a tetraalkylammonium salt.

3. The process of claim 1, wherein the phase transfer catalyst is a tetraalkylphosphonium salt.

4. The process of claim 1, wherein the phase transfer catalyst is a phenol ester derivative of formula (I).

5. The process of claim 2, wherein the tetraalkylammonium salt is a chloride salt.

6. The process of claim 2, wherein the tetraalkylammonium salt is a bromide salt.

7. The process of claim 3, wherein the tetraalkylphosphonium salt is a chloride salt.

8. The process of claim 3, wherein the tetraalkylphosphonium salt is a bromide salt.

9. The process of claim 1, wherein the acid chloride is nonanoyl chloride.

10. The process of claim 1, wherein the acid chloride is octanoyloxyacetyl chloride or nonanoyloxyacetyl chloride.

11. The process of claim 1, wherein the acid chloride is present in molar excess to the phenol sulfonic acid salt.

12. The process of claim 11, wherein the acid chloride is present in a ratio to phenol sulfonic acid salt of at least about 4.5:1.

13. The process of claim 12, wherein the acid chloride is present in a ratio to phenol sulfonic acid salt of at least about 5.5:1.

14. The process of claim 13, wherein the acid chloride is present in a ratio to phenol sulfonic acid salt of at least about 6.7:1.

15. The process of claim 14, wherein the weight ratio of acid chloride to phenol ester derivative (I) in the final product is about 65:35.

16. The process of claim 14, wherein the weight ratio of acid chloride to phenol ester derivative (I) in the final product is about 70:30.

17. The process of claim 14, wherein the weight ratio of acid chloride to phenol ester derivative (I) in the final product is about 75:25.

18. A phenol ester derivative prepared by the process of any one of claims 1-17.

19. A phenol ester derivative of the formula (I)
RCO₂PhSO₃M (I)
where:
R is C₁-C₂₀ linear alkyl; C₁-C₁₅ alkyl substituted by N(R₁)COR₂, CONR₁R₂, CO₂ R₃, OR₃ or SO₂ R₃; OR₃; CH═CHCO₂R₃; phenyl substituted by CO₂R₃; CH(OR₃)₂; CH(SO₂R₃)₂; C(R₄)(R₅)Cl; C(R₇)₂OC(O)R₆; or CH₂OR₈;
R₁ is H or C₁ -C₁₀ alkyl, aryl or alkaryl;
R₂ is C₁-C₁₄ alkyl, aryl or alkaryl;
R₃ is C₁-C₂₀ alkyl, alkenyl, alkynyl or alkaryl, optionally alkoxylated with one or more ethyleneoxy or propyleneoxy groups or mixtures thereof;
R₄ is C₄-C₁₄ alkyl or alkenyl;
R₅ is H, methyl or ethyl;
R₆ is C₁-C₂₀ linear or branched alkyl, alkylethoxyalkylated, cycloalkyl, aryl or substituted aryl;
R₇ are independently H, C₁-C₂₀ alkyl, aryl, C₁-C₂₀ alkylaryl and substituted aryl;
R₈ is aryl optionally substituted by C₁-C₅ alkyl; and
M is selected from an alkali metal or an alkaline earth metal, prepared by reacting an acid chloride of the formula (II)
RCOCl (II)
with a phenol sulfonic acid salt of the formula (III)
HOPhSO₃M (III)
in the absence of solvent, in the presence of from 0.1 to 10 weight percent of a phase transfer catalyst selected from quaternary ammonium and quaternary phosphonium salts and a phenol ester derivative of formula (I), based on the amount of phenol sulfonic acid salt and acid chloride present in the reaction, the phenol ester derivative being a solid porous product having fluid communication to facilitate the passage of gas therethrough.
